(19) United States
(12) Patent Application Publication
(10) Pub. No.: US 2008/0103558 A1
(43) Pub. Date: May 1, 2008

(54) FOCUSED ELECTROMAGNETIC-WAVE AND ULTRASONIC-WAVE STRUCTURES FOR TISSUE STIMULATION

(76) Inventors: Stuart Wenzel, San Carlos, CA (US); Zara Sieh, Pleasanton, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/876,192

(22) Filed: Oct. 22, 2007

Related U.S. Application Data
(60) Provisional application No. 60/863,433, filed on Oct. 30, 2006.

(51) Int. Cl. A61N 1/36

(52) U.S. Cl. 607/61

(57) ABSTRACT

The present invention is directed to small, low profile, antenna-transmitter systems that attach to exterior of the body and focus electromagnetic (EM) wave energy onto one or more precise regions inside the body. The antenna-transmitter system may also deliver energy to the surface of the body without focusing. The present invention is further directed to a method of focusing energy, such as electromagnetic radiation, onto a single nerve to effect selective neurostimulation, super- or sub-threshold, using a small, low profile, antenna-transmitter system that attaches to the body exterior and focuses electromagnetic wave energy onto the nerve.

### CROSS-REFERENCE

This application claims priority from Provisional Application No. 60/863,433 filed Oct. 30, 2006, entitled Focused Electromagnetic-Wave and Ultrasonic-Wave Structures for Tissue Stimulation which application is hereby incorporated herein by reference.

### BACKGROUND OF THE INVENTION

Neurostimulation has had positive clinical outcome for many types of disorders, from chronic pain to Parkinson's disease. This has lead to tremendous interest in practical neurostimulation devices that can be worn by a patient. The major technical challenge has been delivering appropriate electrical energy to one or more precise locations within a body from devices that have minimal invasiveness and high portability.

### SUMMARY OF THE INVENTION

The present invention is directed to small, low profile, antenna-transmitter systems that attach to exterior of the body and focus electromagnetic (EM) wave energy onto one or more precise regions inside the body. The antenna-transmitter system may also deliver energy to the surface of the body without focusing.

The present invention is further directed to a method of focusing energy, such as electromagnetic radiation, onto a single nerve to effect selective neurostimulation, super- or sub-threshold, using a small, low profile, antenna-transmitter system that attaches to the body exterior and focuses electromagnetic wave energy onto the nerve.

### BRIEF DESCRIPTION OF THE FIGURES

The invention will now be described, by way of example only, with reference to the following figures. The accompanying drawings, which are incorporated herein and constitute part of this specification, illustrate presently preferred embodiments of the invention, and, together with the general description given above and the detailed description given below, serve to explain features of the invention, in which:

FIGS. 1A, 1B, and 1C illustrate an embodiment of the present invention that includes electronics, an antenna, and adhesives. The embodiment illustrated in FIGS. 1A, 1B, and 1C can be used to focus electromagnetic waves on particular areas of tissue, such as nerves.

FIGS. 2A and 2B illustrate a parabolic reflector and reflected electromagnetic waves, as can be used in embodiments of the present invention.

FIGS. 3A and 3B illustrate an implanted receiver, as can be used in embodiments of the present invention. The implanted receiver illustrated in FIGS. 3A and 3B receives focused electromagnetic waves and converts them to voltage that can be applied across integrated electrodes.

FIGS. 4A and 4B illustrate another implanted receiver, as can be used in embodiments of the present invention. The implanted receiver illustrated in FIGS. 4A and 4B receives focused ultrasonic waves and converts them to voltage that can be applied across integrated electrodes.

### DETAILED DESCRIPTION OF THE FIGURES

The following detailed description should be read with reference to the drawings, in which like elements in different drawings are identically numbered. The drawings, which are not necessarily to scale, depict selected exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient”, “host” and “subject” refer to any human or animal subject and are not intended to limit the systems or methods to human use, although use of the subject invention in a human patient represents a preferred embodiment.

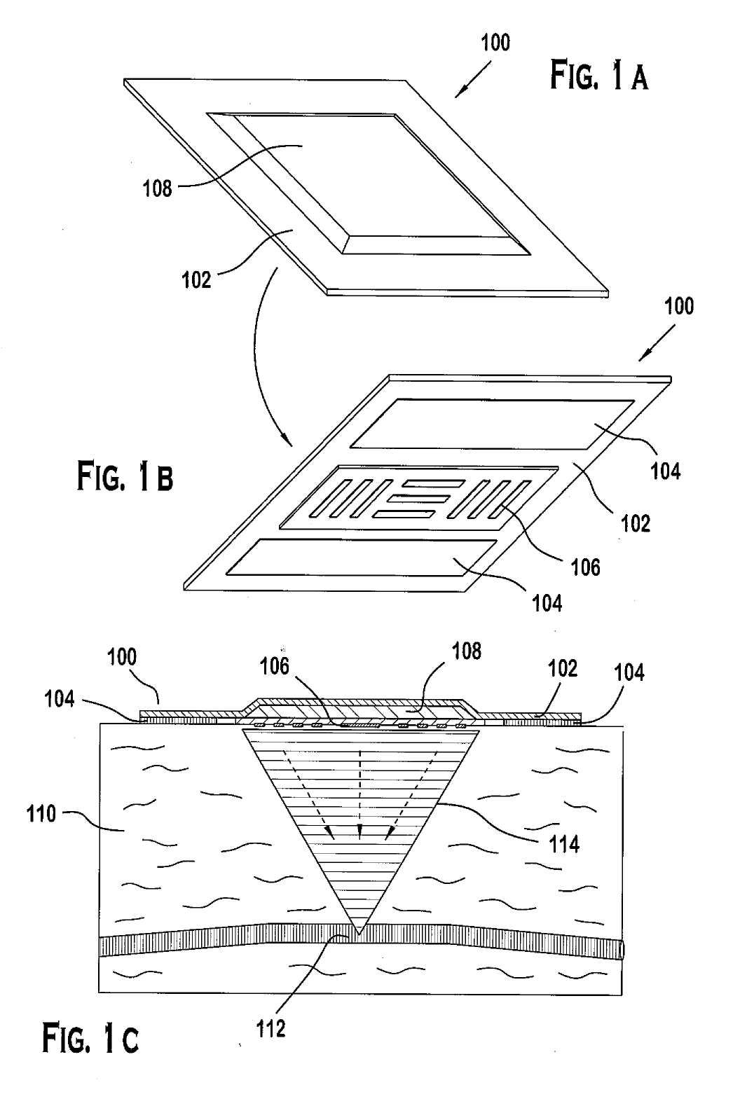

FIGS. 1A, 1B, and 1C illustrate an embodiment of the present invention. FIGS. 1A and 1B illustrate perspective views of patch 100, while FIG. 1C illustrates patch 100 attached to tissue 110. Patch 100 includes antenna array 106 for transmitting and focusing electromagnetic wave 114, electronics 108 for generating an oscillating drive signal for antenna array 106, a power source (part of electronics 108), such as a thin-film or coin-cell battery, and backing 102 that contains the system and allows attachment to tissue 110. Patch 100 also includes adhesive 104 for bonding to tissue 110. Patch 100 enables electromagnetic wave 114 to be focused, while remaining non-invasive. In the embodiment of invention illustrated in FIGS. 1A, 1B, and 1C, energy, in the form of electromagnetic wave 114, passes through tissue 110 and focuses on a specific region, such as nerve 112. Energy in traveling-wave form, such as electromagnetic wave 114, can be focused into regions remote from its source. Electromagnetic waves 114, such as radio waves, microwaves, and visible light, can be transmitted in specific directions using appropriate antennas and/or lenses. In contrast, static electric fields and magnetic fields typically diminish in intensity moving away from their source, and cannot be focused into remote regions.

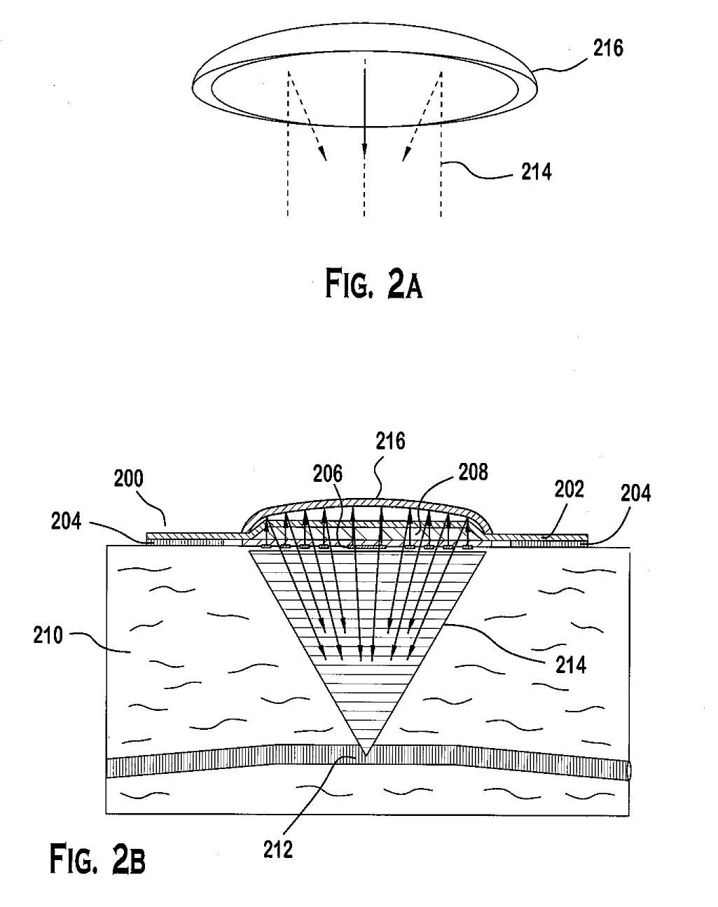

The embodiment of the present invention illustrated in FIGS. 1A, 1B, and 1C includes a micro-strip antenna array 106. Micro-strip antenna array 106 is a particularly good choice for this application, due to its planar structure, small size, and ease of fabrication. In addition, micro-strip antenna array 106 can be fabricated on flexible and/or conformal backings 102. Micro-strip antenna array 106 typically includes conductive traces mounted on substrates with low dielectric loss, such as ceramic or Teflon, and often include a conductive back plane. In further embodiments of the present invention, multiple micro-strip antenna elements can be combined to form phased antenna arrays that direct energy in specific directions. In these embodiments, antenna array 106 and electronics 108 generate traveling waves and can focus high-intensity energy on specific regions. In alternative embodiments of the present invention, other antennas and/or focusing devices may be employed, including slotted micro strip arrays, Fresnel devices, and shaped reflectors (parabolic, spherical, etc). FIGS. 2A and 2B illustrate a parabolic reflector 216 (or reflecting antenna) which can be used to direct and focus electromagnetic wave 214. FIG. 2A illustrates a perspective view of parabolic reflector 216, while FIG. 2B illustrates parabolic reflector 216 integrated into patch 200. In FIG. 2B, patch 200 has been attached to tissue 210. As illustrated in FIG. 2B, electromagnetic wave 214 is emitted by antenna array 206, reflects off parabolic reflector 216, and is focused upon nerve 212 in tissue 210.

In selecting the wavelength of electromagnetic waves 114 or 214, the size and depth of the tissue to be treated should be considered. When focusing energy below the skin surface (or anywhere in space), the minimal focal area typically has a diameter on the order of the wavelength of the wave, in this case electromagnetic waves 114 or 214. Therefore, shorter wavelengths (higher frequencies) result in smaller focused areas, while longer wavelengths (lower frequencies) result in larger focused areas. For example, when using a wavelength of 5 mm, the minimum area of focus is on the order of 5 mm in diameter. If the desired area of treatment is 5 mm in diameter or more, a wavelength of 5 mm can be used. On the other hand, if the desired area of treatment is smaller than 5 mm in diameter, a smaller wavelength may be needed. In addition, antenna arrays 106 and 206 are best focused in far field (typically, more than a few wavelengths away). For this reason, the wavelength affects the focus depth. For example, if one were to use an electromagnetic wavelength of 5 mm, antenna arrays 106 and 206 could best focus at a depth of approximately 20 mm or more.]

In selecting the wavelength of electromagnetic waves 114 or 214, the attenuation of electromagnetic waves 114 or 214 in tissue 110 and 210 should also be considered. There are a variety of causes for attenuation, including absorption, diffusion, and scattering. Absorption, diffusion, and scattering are in many cases a function of wavelength. By shifting from one wavelength to another, attenuation can be dramatically increased or decreased. When electromagnetic waves 114 or 214 are attenuated, more power is required to deliver focused energy on the treatment area. Increased power can result in the need for larger power supplies, and can cause undesirable heating of the tissue surrounding the treatment area. In selecting a wavelength that delivers the best area and depth of focus, one must also consider the wavelength's attenuation. An optimal wavelength allows energy to be focused, while minimizing power consumption due to attenuation.

Returning to FIGS. 1A, 1B, and 1C, electronics 108 would be designed to control the intensity, depth and focal point of electromagnetic wave 114 and might include active devices such as transistors and diodes, and passive devices such as resistors, capacitors and inductors. For higher frequencies (microwave and millimeter-wave), transmission lines (including micro-strip implementations) can be used to form the passive components, and active components can include Gunn diodes, impact ionization avalanche transit-time (IMPATT) devices, monolithic microwave integrated circuits (MMICs), transistors made from silicon and high-speed semiconductors such as GaAs, and other microwave/millimeter-wave devices. Electronics 108 may also include control devices, microprocessors, memory modules, and clocks. Microprocessors, memory modules, and clocks can be combined with algorithms and software to control the functions of patch 100. For example, specific treatment protocols can be programmed where electromagnetic waves 114 and 214 are turned on and off, as desired. In other embodiments, patch 100 may include sensing elements that determine the status of tissue 110 or 210, and vary treatment based on embedded algorithms. Electronics 108 may also include means to communicate with patients and caregivers, such as input keys, displays, and wireless communication devices. Communication means enable the treatment protocol of patch 100 to be modified, and treatment status to be assessed.

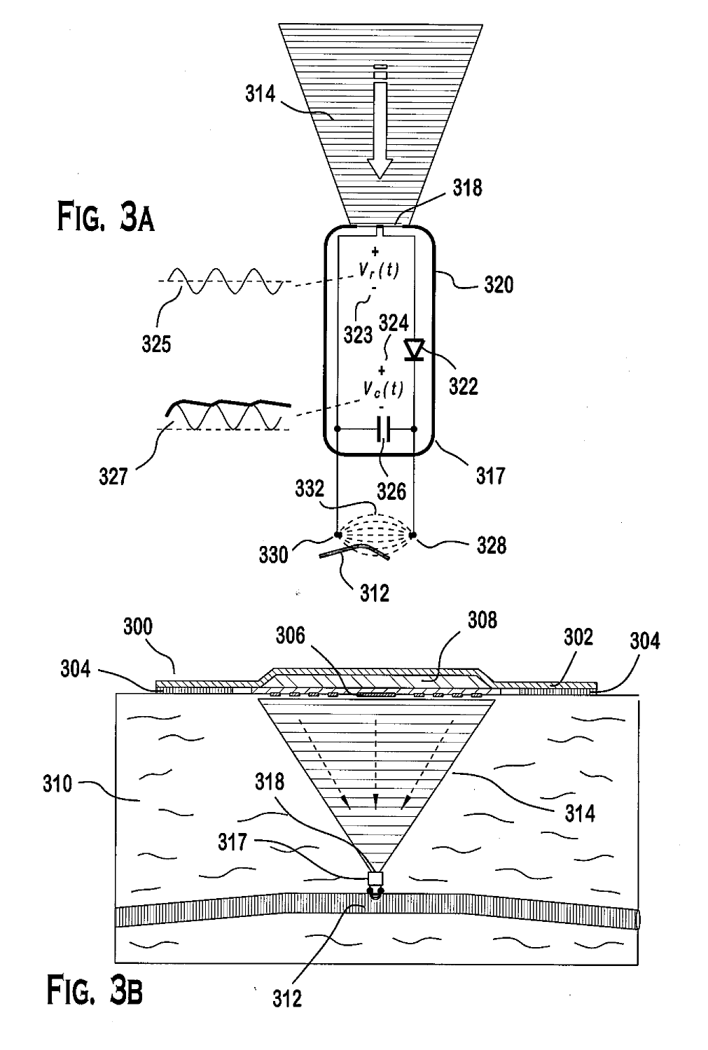

FIGS. 3A and 3B illustrate a further embodiment of the present invention, wherein implanted receiver 317 is used to deliver energy to a particular spot within the body, such as, for example, nerve 312. FIG. 3A illustrates implanted receiver 317 in detail, while FIG. 3B illustrates patch 300 and implanted receiver 317 in tissue 310. Implanted receiver 317 includes enclosure 320, diode 322, capacitor 326, electrodes 328 and 330, and antenna 318. Patch 300 includes backing 302, adhesive 304, antenna array 306, and electronics 308. Backing 302 provides a substrate for various elements, adhesive 304 fixes patch 300 to tissue 310, while antenna array 306 and electronics 308 generate electromagnetic wave 314. In use, implanted receiver 317 is positioned next to the area needing treatment, such as nerve 312. Electromagnetic wave 314 is directed onto receiver antenna 318, where electromagnetic energy is converted to time-dependent received voltage 323 (Vᵣ(t)). Electromagnetic energy is converted to time dependent output voltage 324 (V_{c}(t)) by a half-wave rectifier circuit that includes diode 322 and capacitor 326. Output voltage 324 (V_{c}(t)) is applied across electrodes 328 and 330, in close proximity to the area needing treatment, such as nerve 312. The time-varying voltage 323 (Vᵣ(t)), which is the input to the half-wave rectifier circuit, is illustrated graphically by trace 325, while the time-varying voltage output 324 (V_{c}(t)) of the half-wave rectifier circuit is illustrated graphically by trace 327. In each trace 325 and 327, the horizontal axis represents time and the vertical axis represents voltage. Implanted receiver 317 may be about 1 mm in diameter by about 1-3 mm long, as an example. Implanted receiver 317 rectifies the electromagnetic wave 314 before delivering its energy to tissue, or nerve 334. An advantage of using an implanted receiver, as illustrated in FIGS. 3A and 3B, is that less spatial precision is needed when focusing an electromagnetic wave. The implanted receiver can be very small, and positioned very accurately, delivering its energy to a precise location while receiving its power from a wide electromagnetic wave. The implanted receiver illustrated in FIGS. 3A and 3B can be made with tiny, discrete components, integrated into a hermetic package (ceramic, metal such as titanium) that is less than a few millimeters in diameter and length. In on& embodiment of the present invention, the integrated component assembly is encased in silicon carbide, creating a long-term hermetic seal.

Generally, the most effective electrical signals for electrostimulation are pulsed, with repetition rates around 0.1-100 Hz. The electromagnetic waves discussed herein have a much higher frequency (called the carrier frequency), and will generally be gated on and off with the electrostimulation signal in the aforementioned frequency range; the electrostimulation signal therefore forms the modulation envelope of the carrier. When an implanted receiver is used, as illustrated in FIGS. 3A and 3B, the receiver “strip” the carrier and replicate the envelope of the carrier, meaning that the output voltage will be the desired electrostimulation signal. For example, tissue stimulation might require a square wave that has 1 ms on and 10 ms off, while the electromagnetic waves might have a carrier frequency of 500 MHz. The transmitted electromagnetic signal will be modulated on and off with the 1 ms/10 ms electrostimulation signal; then the receiver will strip the 500 MHz carrier signal and produce the 1 ms/10 ms square wave on the output V_{c}(t).

In further embodiments of the present invention, focused ultrasonic energy may be used to provide stimulation at particular points within tissue. Ultrasonic devices can be made that focus energy to precise locations within tissue. These can produce ultrasonic stimulation directly, as well as electrical fields indirectly, by taking advantage of piezoelectric properties that are inherent in certain types of tissue cells. One advantage of ultrasonic waves over electromagnetic waves is that ultrasonic waves have lower wave velocity, resulting in shorter wavelengths and more precise focusing. At a wavelength of 1 mm, ultrasound frequency is about 1.5×10⁶ Hz, while electromagnetic wave frequency is about 3×10¹¹ Hz. In general, higher frequencies require more complex electronics.

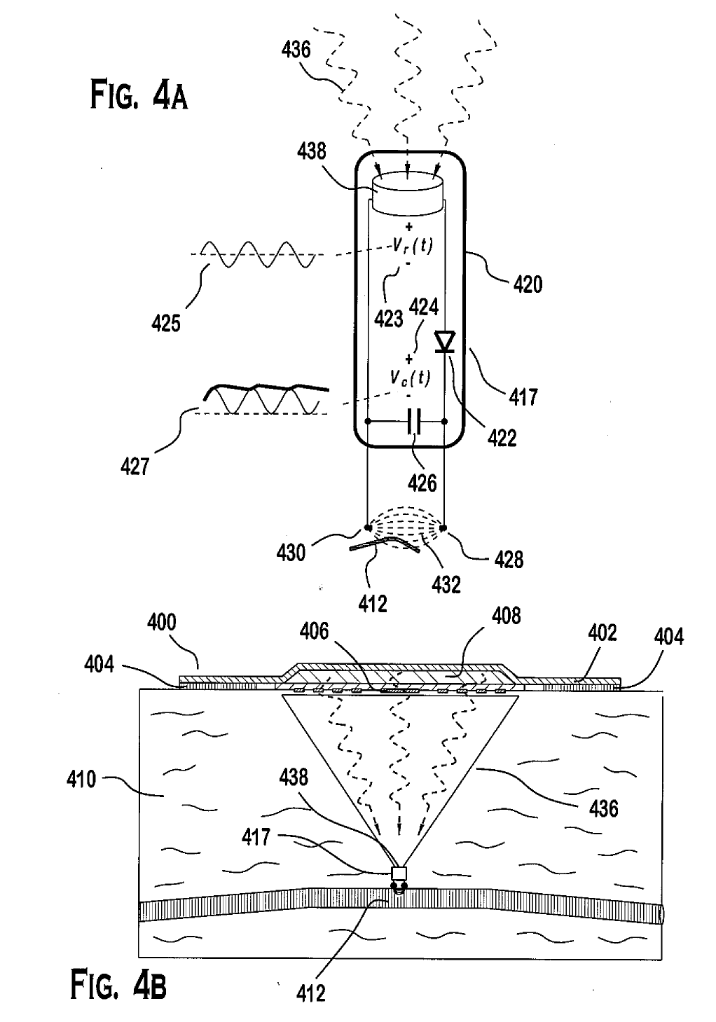

An embodiment of the present invention that uses focused ultrasound is illustrated in FIGS. 4A and 4B. FIG. 4A illustrates implanted receiver 417 in detail, while FIG. 4B illustrates patch 400 and implanted receiver 417 in tissue 410. Implanted receiver 417 includes enclosure 420, diode 422, capacitor 426, electrodes 428 and 430, and transducer 438. Patch 400 includes backing 402, adhesive 404, ultrasonic transmitters 406, and electronics 408. Backing 402 provides a substrate for various elements, adhesive 404 fixes patch 400 to tissue 410, while ultrasonic transmitters 406 and electronics 408 generate ultrasonic wave 436. In use, implanted receiver 417 is positioned next to the area needing treatment, such as nerve 412. Ultrasonic wave 436 is focused on transducer 438, where ultrasonic energy is converted to time-dependent input voltage 423. From voltage 423, ultrasonic energy is converted to time dependent output voltage 424 by a half-wave rectifier circuit that includes diode 422 and capacitor 426. Voltage is applied across electrodes 428 and 430, in close proximity to the area needing treatment, such as nerve 412. The time-varying received voltage, which is the input to the half-wave rectifier circuit, is illustrated by input voltage trace 425, while the time-varying voltage output of the half-wave rectifier circuit is illustrated by output voltage trace 427. A preferred embodiment of the present invention includes suitable transducer materials to perform the acoustic-to-electric conversion, such as piezoelectric materials (PZT, ZnO, AlN, polyvinylidene fluoride (PVDF, PVF₂)), as well as electrodes to collect and deliver electrical energy (charge) to the tissue. Passive electrical components such as diodes and capacitors may also be included to rectify and collect power, converting AC acoustic power to DC electrical power. Focused ultrasonic transmitters, as used in the present invention, can be built using medical ultrasonic transceivers.

Implanted receivers 317 and 417 may also include energy storing devices, control devices, microprocessors, memory modules, and clocks. Energy storing devices include batteries and capacitors. Batteries can be installed prior to implantation; while energy storing capacitors can be charged in vivo. In vivo charging can be accomplished using electromagnetic waves transmitted through tissue and collected by on-board components, such as super capacitors. Microprocessors, memory modules, and clocks can be combined with algorithms and software, controlling the functions of implanted receivers 317 and 417. For example, specific treatment protocols can be programmed to deliver energy that varies as a function of time, allowing output voltages 324 and 424 to be turned on and off, as desired. In other embodiments, implanted receivers 317 and 417 may include sensing elements that determine the status of the treatment area, and vary treatment based on embedded algorithms. Implanted receivers 317 and 417 may also include means to communicate with patients and caregivers, such as input keys, displays, and wireless communication devices. Communication means enable the treatment protocol of implanted receivers 317 and 417 to be modified, and treatment status to be assessed.

A key advantage of devices according to the present invention is that they are non-invasive, portable, easy to wear, and disposable. They can also be relatively low cost.

While preferred embodiments of the present invention have been shown and described herein, it will be obvious to those skilled in the art that such embodiments are provided by way of example only. Numerous variations, changes, and substitutions will now occur to those skilled in the art without departing from the invention. Various alternatives to the embodiments of the invention described herein may be employed in practicing the invention. It is intended that the following claims define the scope of the invention and that methods and structures within the scope of these claims and their equivalents be covered thereby.

What is claimed is:

1. A tissue stimulation device adapted to be affixed to the body of a patient, said device comprising:
a power source;
electronics for generating a signal;
an energy transmitter structure;
a receiver; and
a substrate supporting the power source, electronics and energy transmitter structure, wherein said substrate is adapted be affixed to the outside surface of a patient and the receiver is adapted to be implanted below the skin in proximity to tissue to be treated and is further adapted to receive energy from said tissue stimulation device and convert said energy into a signal for stimulating said tissue.

2. A tissue stimulation device according to claim 1 wherein said energy comprises a source of electromagnetic radiation.

3. A tissue stimulation device according to claim 2 wherein said energy transmitter comprises an electromagnetic antenna such as a micro-strip antenna array, a fresnel device, a shaped reflector or a combination of these.

4. A tissue stimulation device according to claim 3 wherein said antenna structure comprises a micro-strip antenna array.

5. A tissue stimulation device according to claim 1 wherein said energy comprises a source of ultrasonic energy.

6. A method of stimulating tissue by the transmission of energy through the skin of a patient, said method comprising the steps of:
positioning a tissue stimulation device on a surface of the skin of a patient to be treated, wherein the tissue stimulation device comprises a power source, electronics for generating a signal, a transmitter structure and a substrate supporting these that is adapted be affixed to the skin of a patient;
transmitting focused energy to a localized spot or region below the surface of the skin; and
converting said transmitted focused energy to a different form using a receiver positioned below the surface of the skin proximal to the tissue to be treated.

7. A method of stimulating tissue according to claim 6 wherein said transmitted focused energy comprises a source of electromagnetic energy.

8. A method of stimulating tissue according to claim 6 wherein said transmitted focused energy comprises a source of ultrasonic energy.

9. A tissue stimulation device according to claim 5 wherein said energy transmitter comprises an ultrasonic transmitter such as a disk of piezoelectric material.
